(19) United States
(12) Patent Application Publication
(10) Pub. No.: US 2022/0262457 A1
(43) Pub. Date: Aug. 18, 2022

(54) A DEEP LEARNING APPROACH TO CORRELATE CELLULAR MORPHOLOGY AND GENETICS

(71) Applicants: International Business Machines Corporation, Armonk, NY (US); The Regents of the University of California, Oakland, CA (US)

(72) Inventors: Vito Paolo Pastore, Lecce (IT); Simone Bianco, San Francisco, CA (US); Wallace Marshall, San Francisco, CA (US)

(21) Appl. No.: 17/175,495

(22) Filed: Feb. 12, 2021

(51) Int. Cl. G16B 20/00; G16B 40/00; G06N 3/08; G06K 9/62

(52) U.S. Cl. CPC G16B 20/00; G06K 9/6262; G06N 3/08; G16B 40/00

(57) ABSTRACT

Provided is a data-driven deep-learning based algorithm for synthetic biology applications that makes no assumptions and/or hypotheses on genotype-phenotype interactions. deep-learning based algorithm trains a neural network with morphological features from single genetic modifications and tests the neural network with morphological features from multiple genetic modifications. The trained and tested neural network uses a link between the morphological features caused by the single and multiple gene modifications as input and outputs a genotype-phenotype mapping highlighting perturbation subspaces. The genotype-phenotype mapping is used to select one or more genetic insults as a starting point to engineer cells in synthetic biology applications.

### STATEMENT REGARDING FEDERALLY SPONSORED RESEARCH OR DEVELOPMENT

This invention was made with Government support under DBI-1548297 awarded by the National Science Foundation. The Government has certain rights to this invention.

### TECHNICAL FIELD

The present invention relates to artificial intelligence programs and more specifically to a deep-learning based algorithm for synthetic biology applications that correlates the relationship between cellular morphologies and potential genetic insults that may result in the cellular morphologies.

### BACKGROUND

Synthetic biology is a growing billion-dollar market. With synthetic biology, cells are genetically engineered to produce a wide range of products, such as biofuels, drugs, antibodies, cosmetics, flavorings, and scents. Synthetic biology products are typically produced through a trial-and-error process where cells are subject to targeted combinations of genetic modifications until the expected product is obtained. The spatial heterogeneity in the individual cell response is determined through ordinary differential equations (ODE), which do not always provide accurate representations of the cell signaling dynamics that occur as a result of the targeted genetic modifications. An ODE that does not accurately reflect the cellular response that occurs during the genetic modification can result in a failed synthetic biology engineering process. The uncertainty resulting from the use of ODEs to determine cellular responses coupled with the resources and time required to implement the trial-and-error process results in synthetic biology methods that can be costly and inefficient. There is a need in the art for a synthetic biology engineering approach that applies a more specific approach to cellular response modeling and is not reliant on trial and error to produce synthetic biology products.

### SUMMARY OF THE INVENTION

The present invention overcomes the limitations in the art by providing a data-driven deep-learning based algorithm for synthetic biology applications that makes no assumptions and/or hypotheses on genotype-phenotype interactions.

In one embodiment, the present invention relates to a computer-implemented method for genotype-phenotype mapping for single and multiple genetic insults comprising: training a deep learning neural network with cellular morphology features from single genetic modifications; testing the deep learning neural network with cellular morphology features from multiple genetic modifications, wherein the trained and tested deep learning neural network inputs a link between cellular morphology features caused by the single gene modifications and cellular morphology features caused by the multiple gene modifications and outputs a genotype-phenotype mapping highlighting perturbation subspaces.

In another embodiment, the present invention relates to a computer program product for genotype-phenotype mapping for single and multiple genetic insults comprising: one or more computer readable storage media, and program instructions collectively stored on one or more computer readable storage media, the program instructions comprising: program instructions for training a deep learning neural network with cellular morphology features from single genetic modifications; program instructions for testing the deep learning neural network with cellular morphology features from multiple genetic modifications; and program instructions for the trained and tested deep learning neural network to input a link between cellular morphology features caused by the single gene modifications and cellular morphology features caused by the multiple gene modifications and output a genotype-phenotype mapping highlighting perturbation subspaces.

In one aspect, the present invention relates to a method comprising: inducing single gene modifications in a first portion of a cell sample and obtaining a first set of cell images; inducing multiple gene modifications in a second portion of the cell sample and obtaining a second set of cell images; extracting cellular morphology features from the first and second set of cell images; training a deep learning neural network with the cellular morphology features from the first set of cell images; testing the deep learning neural network with the cellular morphology features from the second set of cell images, wherein the trained and tested neural network applies the cellular morphology features from the first and second set of images as input and provides genotype-phenotype mapping highlighting perturbation subspaces.

Additional aspects and/or embodiments of the invention will be provided, without limitation, in the detailed description of the invention that is set forth below.

### BRIEF DESCRIPTION OF THE DRAWINGS

FIG. 1 is a flowchart showing application of the data-driven deep learning unboxing algorithm on a neural network with single and multiple genetic modifications as input and a genotype-phenotype mapping as output.

FIG. 2 is a graph showing genotype-phenotype mapping of a cellular morphology space showing the subspaces of viable genetic modifications and useful cellular morphology phenotypes.

FIG. 3 is a block diagram showing the components of an exemplary neural network system with a cellular input and a genotype-phenotype mapping output.

FIGS. 4A and 4B are representative data matrices that may be generated by the deep learning unboxing algorithm. FIG. 4A is an example of a classification matrix for misclassified samples and FIG. 4B is an example of a misclassified data range matrix derived from the classification matrix for the misclassified samples.

### DETAILED DESCRIPTION

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein, the term “genetic modification” is used to refer to the manipulation of the genome of a cell through induced mutations. Induced mutation may include large-scale mutations in chromosomal structure that alter gene expression and small-scale mutations of one nucleotide (single point mutations) or a few nucleotides that change the function of genes. Examples of large-scale mutations include, without limitation, amplification or repetition of a chromosomal segment (leading to an increase in the genes within the chromosomal region), deletion of chromosomal regions (leading to loss of the genes within those regions), chromosomal rearrangement (leading to a decrease in gene fitness), chromosomal translocations (interchange of genetic parts from nonhomologous chromosomes), chromosomal inversions (reversing the orientation of a chromosomal segment), non-homologous chromosomal crossover (the pairing up of chromatids from non-homologous chromosome pairs), interstitial deletions (intra-chromosomal deletion that removes a segment of DNA from a single chromosome causing previously distance genes to become apposed), and loss of heterozygosity (the loss of one allele of an allele pair through deletion or genetic recombination). Examples of small-scale mutations include, without limitation, insertions (adding one or more nucleotides into the cell DNA), deletions (removing one or more nucleotides from the cell DNA), and substitutions (exchange of a single nucleotide for another). Within the context of the present invention, genetic modifications include both single gene modifications and multiple gene modifications.

As used herein, the term “genetic insults” refers to one or more events that alter the DNA of a cell resulting in mutations within the genetic material of the cell. Genetic insults are typically genetic or environmental. Within the context of the present invention, induced mutations through genetic modification result in genetic insults.

As used herein, the term “genetic distance” refers to the number of differences or mutations between two samples, such as a source sample and a naturally or synthetically mutated sample derived from the source sample. A genetic distance value of zero means that there are no differences between the two samples.

As used herein, the term “cellular morphology” refers to all aspects of a cell, including all external and internal morphology. External morphology relates to the outward appearance of the cell and includes, without limitation, the shape, structure, color, size, and pattern of the external features of the cell. Internal morphology relates to the internal anatomy of a cell and includes, without limitation, the form, structure, and arrangement of the internal bones, organs, and organelles of the cell.

As used herein, the terms “perturbation” and “cellular morphology perturbation” are used interchangeably to refer to the physical alteration of a cell that has undergone one or more genetic modifications and/or displays one or more genetic insults.

As used herein, the term “genotype-phenotype mapping” refers to the correlation of genetic factors to phenotypic trait variation. A typical genotype-phenotype map pairs each genotype to one or more phenotypes. Within the context of the present invention, genotype-phenotype mapping pairs the metrics of genetic distance to perturbations.

As used herein, the term “subspace” refers to a statistical classification approach where each class is represented and modeled by a subspace, which is lower in dimension from the original space. A subspace of each class may overlap with each other or may be mutually exclusive. Within the context of the present invention, the term “perturbation subspaces” refers to the classification of one or more phenotypic perturbations that are identified and/or data mined from a larger space of genetic-morphological factors.

As used herein, the term “deep learning” refers to an artificial intelligence (AI) function that mimics the workings of the human brain in processing data. Deep learning AI is able to learn without human supervision drawing from data that is unstructured and unlabeled. Deep learning is a subset of “machine learning,” which is an AI function that uses algorithms to parse data, learn from that data, and make informed decisions based on what was learning. Deep learning differs from machine learning in that the former mimics human-like AI while the latter does not.

As used herein, the term “neural network” refers to a deep learning classification algorithm that can take in an input image, assign importance (learnable weights and biases) to various aspects/objects in the image and be able to differentiate one aspect/object in the image from another. The architecture of a neural network is analogous to that of the connective pattern of neurons in the human brain; thus, neural networks use the terminology “neurons” to refer to each mathematical operation within the neural network.

As used herein, the term “unboxing algorithm” refers to an AI algorithm that uses a “black box” model, which is a model that applies one or more layers of machine and/or deep learning decisions based on a set of rules or parameters without human supervision. Within the context of the present invention, an unboxing algorithm may be used as a deep learning classification algorithm within a neural network.

As used herein, the term “reverse engineering” as it applies to a trained and tested neural network refers to an unboxing algorithm that analyzes output to prune insignificant neurons and identify significant neurons that lead to the output. One example of a reverse engineering algorithm is the RxREN, which uses an IF/THEN extraction rule to classify the output from a neural network.

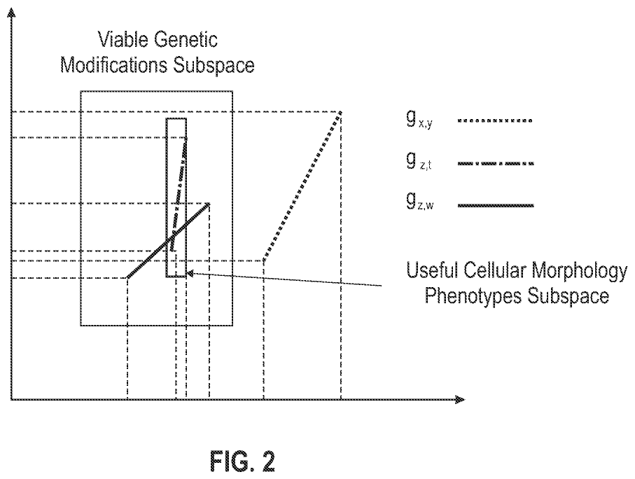

Described herein is a data-driven deep-learning based unboxing algorithm for synthetic biology applications that reconstructs the genotype-phenotype mapping of a cellular organism to correlate the relationship between cellular morphologies and potential genetic insults that may result in cellular morphology perturbations. The unboxing algorithm first uses a neural network to learn the non-linear model representing the cellular morphology perturbations resulting from single local genetic insults and then incorporates multiple genetic insults to reveal cellular morphology perturbation patterns that are common between the single and multiple genetic insults leading to the genotype-phenotype mapping. As shown in FIG. 2, the genotype-phenotype mapping can provide a set of ranges for cellular morphology inferred attributes as a function of a specific sequence of cellular genetic insults while highlighting genetic distance and/or perturbation subspaces, such as for example, viable genetic modifications and/or useful cellular morphology phenotypes. Using the metrics of genetic distance and perturbance, a genetic insults starting point for engineering cells for a select synthetic biology application can be determined.

Unlike synthetic biology models currently used in the art, the unboxing algorithm makes no assumptions and/or hypotheses on genotype-phenotype interactions and does not apply ODEs to model the cellular responses that occur from genetic modifications. By defining a space of genetically correlated cellular morphology features, the unboxing algorithm provides an efficient starting point for synthetic biology engineering processes by avoiding the implementation of genetic modifications that could lead to non-viable phenotypes. Further, by providing a subset of viable genetic modifications that lead to useful phenotypes, the unboxing algorithm excludes modifications to specific genes that result in trivial or useless phenotypes, such as for example, genetic modifications that could result in reduced cellular viability, survival, and/or production.

The unboxing algorithm can additionally provide a set of AI primitives that can be used to support synthetic biology engineering design processes. For example, upon the generation of sufficient data correlating certain genetic insults with useful phenotypes, a computer aided design system can be built to guide genetic engineering for a specific synthetic biology product. The unboxing algorithm may additionally define a closed loop system capable of facilitating genetic modification by controlled cellular morphology perturbations.

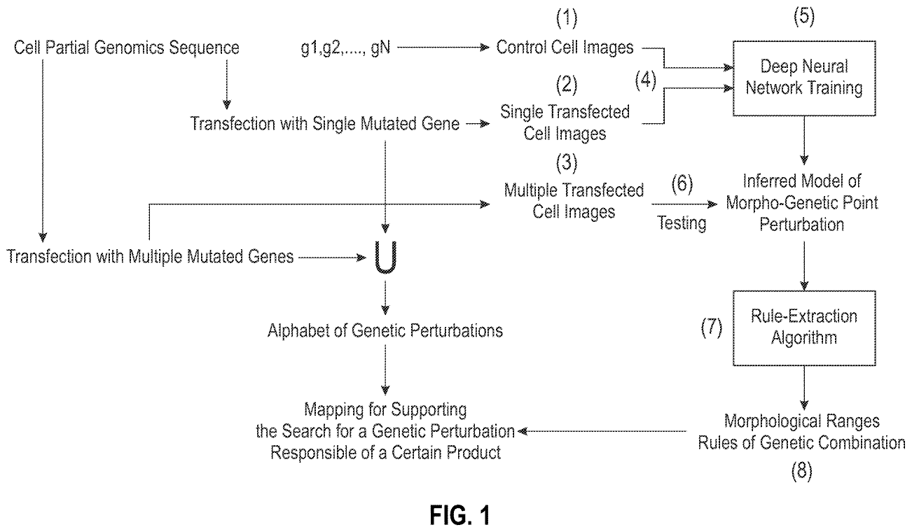

FIG. 1 shows a representative workflow for the deep-learning based algorithm described herein, which includes the following steps:
(1) From a cell sample, a cellular genetic sub-sequence representing a set of experimentally possible genetic modifications as represented by >g1,g2, . . . , gN is established and multi-channels control images (i.e., empty vector images) are acquired from the cells.
(2) Single gene modifications are induced in the cells and corresponding cell images are acquired.
(3) Multiple gene modifications (as a combination of the single modifications) are induced and corresponding cell images are acquired where the output is a set of cell images showing multiple genetic modifications and the corresponding images for the single perturbations.
(4) A set of morphological features are extracted from the output images of steps (2) and (3), including all of the available organelles, where the morphological features are shape descriptors for the cell that need to be engineered according to the type of cells.
(5) Single gene perturbation and control images are used to train a deep neural network.

The training allows the network to learn a non-linear function that separates the controls (i.e., the cells that have not been genetically altered) from the transformed cells. Multiple neural networks are trained according to the sequences obtained following the multiple gene modification of step (3).
(6) A test dataset, represented by the features extracted from the multiple gene modifications that correspond to the single genes, is used to train each of the neural networks from training step (5). The testing data is classified as belonging to the transformed cells with the deep neural network using the training data to classify the multiple transformed cells as mutated rather than as controls.
(7) The trained and tested neural networks are black boxes that take in the morphological features of the cells as input and provide the cell classification as output. A reverse engineering automated rule-extraction algorithm (e.g., RxREN) is used to construct an unboxing algorithm that unboxes the neural network and reveals the ranges among the morphological attributes that the neural network finds to be similar between the single and multiple transformed cells.
(8) The output after step (7) is a set of input ranges that link the morphology of single and multiple genetic insults where the input ranges map the genetic space into the morphological space. FIG. 2 shows the mapping of (i) an input range of viable genetic modifications imposing morphological restraints (genetic modifications g_{z,w}) and (ii) an input range of viable genetic modifications that are most likely to result in a useful cellular morphology phenotype (genetic modifications g_{z,t}) (the genetic modifications g_{x,y} represent non-viable genetic modifications).

With reference to steps (1)-(8), once a sequence of cell images is acquired, features design and segmentation algorithms are applied as needed. For example, confocal fluorescence images on spinning disks may be used for features design and segmentation algorithms may be applied that are based on automatic and manual intensity pixel thresholding. Examples of cellular morphology features that may be used in the design include shape attributes, including, without limitation, perimeter of a cell, area of a cell, nucleus circularity, convexity, and number of mitochondria. For the neural network described herein, the input layer has as many neurons as the number of extracted features with at least one hidden layer with x neurons and an output layer with two classes: mutated cells and control cells. In one embodiment, the number of hidden layers is one. In another embodiment, the number of hidden layers is two. In a further embodiment, the number of hidden layers is in the range of one to five. The range for the x neurons will depend on the dataset. In one embodiment, the range of x neurons in the at least one hidden layer is 100-1000 neurons. In another embodiment, the range of x neurons in the at least one hidden layer is 100-500 neurons. In a further embodiment, the range of x neurons in the at least one hidden layer is 100-250 neurons. In another embodiment, the number of hidden layers is two and the number of x neurons in the two hidden layers is 100. Once the trained model is obtained, the automated rule-extraction algorithm is used to construct the unboxing algorithm through input pruning, data range computation, rule extraction, rule pruning, and rules update.

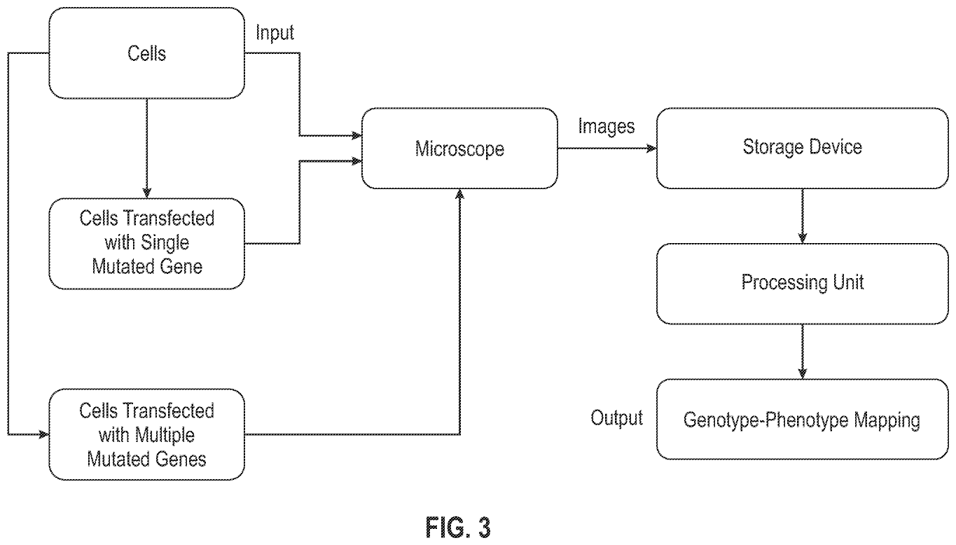

FIG. 3 shows an exemplary neural network system with a cellular input and a genotype-phenotype output. As shown therein, the neural network system includes the physical components of a microscope that produces images from a cellular input, a data storage device for storing the images, and a computer processing unit that applies the algorithm to produce the genotype-phenotype mapping output.

### Input Pruning

The first step in the construction of the unboxing algorithm comprises pruning neurons corresponding to input features that do not significantly affect the network's accuracy. For a set of N input neurons n1,n2, . . , nN, to assess if a neuron ni is not significant, the neuron ni is eliminated by setting its value to 0 and obtaining the input sequence n1, . . . , ni−1,0,ni+1, . . . , nN. Next, the network's corresponding classification error Ei is computed on a testing subset of data. The procedure is equally iterated for i ∈[1,N]. If Ei<ϑ|ϑ=miniEi, then the neuron ni is not significant and it is pruned. The accuracy of the pruned network is set at PNacc and the accuracy of the original trained network is set at ONacc. The foregoing pruning procedure is iterated until the condition PNacc≥σ*ONacc is verified, where σ is a parameter representing the allowed drop in accuracy consequent to the pruning procedure. For purposes of illustration, a value of σ=0.99 indicates as maximum drop in accuracy of 1%.

### Data Range Computation

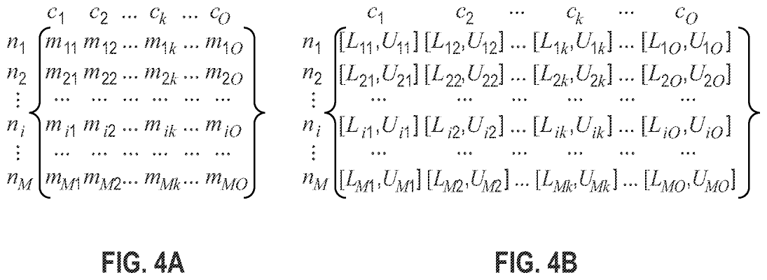

For the data range computation, p1,p2, . . , pM, M≤N represents the pruned set of input features pi resulting from the input step. First, the contribute of the input feature pi is eliminated by setting its value to 0 and obtaining the input pruned sequence p1, . . . ,pi−1,0,pi+1, . . . ,pN. Second, the trained neural network is used to compute mik, where mik is the number of misclassified testing samples belonging to output class k corresponding to the removal of input feature pi and where |∈[1,M], k ∈[1,O], M is the number of pruned input neurons, and 0 is the number of output neurons (and classes). Third, from within the misclassified samples mik, the minimum Lik and maximum Uik values are computed among the misclassified samples mik to produce a misclassified data range matrix Dik. FIG. 4A shows a matrix of misclassified samples according to the second step of the data range computation and FIG. 4B shows a misclassified data range matrix Dik according to the third step of the data range computation.

The trained neural network does not necessarily make use of all the input features to classify a specific pattern into a specific class. For example, one input feature may not be required to correctly classify the data into all the output classes, but it could be fundamental for the classification into a specific class k. To construct a set of rules equivalent to a trained neural network, input features that are unnecessary to classify a certain class are excluded from the data range computation; thus, the number of misclassified samples matrix mik only includes significant neurons for each of the output classes. The cumulative (i.e., sum over the output classes) number of misclassified samples after the removal of input neuron ni is denoted as mi_total where the input i is considered significant with respect to class k if and only if mik>α mi_total, where α ∈[0.1,0.5] is a threshold parameter representing the minimum fraction of misclassified samples required for considering neuron ni significantly impacting on the discovery of class k. If such condition is not verified, the input i is excluded from the rule construction of class k, and the data range matrix correspondent entry Dik is put to zero as provided in Formula (1):

$${D}_{i k} = \{ \begin{matrix}[ {L}_{i k} , {U}_{i k} ] , & {m}_{i k} > α {m}_{i \_ total} \\ 0 , & otherwise\end{matrix} . \qquad ( 1 )$$

### Rule Extraction

For the rule extraction, the set of rules is directly extracted from the columns of Dik. Each column k of the computed data range matrix Dik represents the range of input features that the trained neural network requires to classify a pattern as belonging to class k. A zero entry corresponds to input features not necessary for the classification into class k. The higher the number of a column k non-zero entry, the more restrictive is the corresponding rule. In a descending order, starting from the class that requires the highest number of input features for classification, a rule is defined for each of the output classes. For a generic class k, a rule is constructed according to Formula (2):

IF (L1k<n1≤U1k & L2k≤n2≤U2k & . . . & LMk≤nM≤UMk)

THEN class=ck (2)

The extracted set of rules is equivalent to the trained neural network and can be used to perform classification based on the range of significant input features.

### Rule Pruning

The rule pruning step removes unnecessary conditions from each of the defined rules. A condition cndi=Lik≤ni≤Uik is considered not required if the accuracy of the total set of rules increases or is not affected by its removal. First, the classification's accuracy Rulesacc is evaluated on the testing data using the extracted set of rules. Then, for each of the constructed rules rk, the accuracy R_pruned_k_iacc of the rule is computed by the removal of each of the conditions cndi; thus, if Rulesacc≤R_pruned_k_iacc, the condition is removed. The procedure is iterated for all the conditions defining each of the constructed rules.

### Rules Update

The last step in the construction of the unboxing algorithm comprises improving the classification accuracy of the pruned set of rules. With reference to the data range matrix Dik, which extracts the minimum and maximum value of the trained neural network's misclassified samples, some of the computed input ranges may overlap between different classes with a consequent decreasing in the classification accuracy. To improve the classification accuracy, the data range matrix Dik is updated according to the misclassified samples resulting from the constructed set of rules where a specific rule condition is updated only if the update corresponds to a classification accuracy increase. For example, Lrik and Urik are the minimum and the maximum values, respectively, of the misclassified samples corresponding to the condition cndi in rule rk. The rule cndi is updated to replace the condition Lik≤ni≤Uik with Lrik≤ni≤Urik, if and only if the classification accuracy corresponding to the update set of rules is higher or equal to the classification accuracy of the original rule. The procedure is iterated among all of the conditions for all of the extracted rules.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, a graphics processing unit (GPU) (for deep learning), programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### EXPERIMENTAL

The following examples are set forth to provide those of ordinary skill in the art with a complete disclosure of how to make and use the aspects and embodiments of the invention as set forth herein. While efforts have been made to ensure accuracy with respect to variables such as amounts, temperature, etc., experimental error and deviations should be considered. All components were obtained commercially unless otherwise indicated.

### EXAMPLE 1

### Establishing an Image Dataset for Mouse Embryonic Fibroblasts (MEFs) (Steps 1-3)

An example dataset was developed using spinning disk confocal fluorescence images of mouse bladder embryonic fibroblasts (MEFs). MEFs were transfected with the oncogenic mutated genes H-Ras and Myc two weeks after conception. The H-Ras gene is located on the short (p) arm of chromosome 11 and has shown to be a proto-oncogene whose specific mutations have been associated to bladder cancer. Cells were fixed and stained, and images of transformed cells were acquired two weeks after mutation. Cellular transformation was tested using an agar penetration assay. The resulting dataset included images of: (i) cells transfected with an empty vector, used as control; (ii) cells transfected with mutated H-Ras only; (iii) cells transfected with mutated Myc only; and (iv) cell transfected with both oncogenic mutated H-Ras and Myc. The cells were stained with the following four different fluorescent dies to highlight four different cellular structures and organelles: (1) nucleus (DAPI, blue), (2) nucleoli (TRITC/anti-fibrillarin, white), (3) mitochondria (FITC/anti-mtHSP70, green), and (4) actin (A647-Phalloidin, red), which is a proxy for the cell body. The cell images (i)-(iv) were pre-processed using common preprocessing steps. For example, a gaussian blurring filter was used to reduce the effect of image noise on segmentation. Each adopted algorithm was based on a pixel intensity thresholding method, where a pixel was set to OFF if its intensity was lower than a computed threshold and was otherwise set to ON. Both global and local approaches were used for pixel intensity thresholding. With the global thresholding algorithm, a fixed threshold was first computed and then applied to the pixels of the entire image. With the local or adaptive methods, a threshold was computed locally in a fixed size neighborhood of each pixel of the image, resulting in a different threshold applied in different regions of the image.

### EXAMPLE 2

### Segmentation of the MEF Cell Images and Features Extraction (Step 4)

The MEF images from Example 1 were segmented and organelles segmentation was further extracted for quantification. To do this, each cell segment was described by a different set of features, each appropriate for the general description of an organelle's state as it related to the state of the cell. Examples of the segmented cell features included cell perimeter, area, organelle number (total and average), nucleus average perimeter and area, eccentricity, roundness and circularity. A total of 26 features were extracted.

### EXAMPLE 3

### Designing the Neural Network (Steps 5 & 6)

From the 26 extracted features of Example 2, the following were designed: (i) a neural network with an input layer of 26 neurons (one per each engineered feature); (ii) an output layer with two neurons (corresponding to the two classes, controls and mutations); and (iii) two hidden layers (40 neurons). The network was trained for 120 epochs using the arrays of the 26 extracted features. Two classes were used for training: controls and single transformed cells. The training accuracy reached 98% as well as a validation accuracy of 97%. When the neural network was tested using the double transformed extracted features, an accuracy of around 78% was reached.

### EXAMPLE 4

### Establishing the Unboxing Algorithm and Establishing a Set of Rules Corresponding to the Cell Morphologies (Steps 7 & 8)

The neural network of Example 3 was unboxed using an RxREN algorithm. The RxREN algorithm adopted a reverse engineering approach to extract rules from the trained neural network based on a subset of the testing data. A pruning of input neurons was used to reduce the dimensionality of the input data. Next, the misclassified training data (i.e., the training data whose assigned label is not correct) were exploited to infer the set of rules that allow the neural network to assign each of the possible output classes. The customized version of the RxREN algorithm consisted of five different phases: (1) input pruning, (2) input range computation, (3) rule definition, (4) rule pruning, and (5) rules update. Finally, a set of rules were established correlating specific input cellular morphologies to the H-Ras and Myc modifications most likely responsible for the cellular morphology.

We claim:

1. A computer-implemented method for genotype-phenotype mapping for single and multiple genetic insults comprising:
training a deep learning neural network with cellular morphology features from single genetic modifications;
testing the deep learning neural network with cellular morphology features from multiple genetic modifications,
wherein the trained and tested deep learning neural network inputs a link between cellular morphology features caused by the single gene modifications and cellular morphology features caused by the multiple gene modifications and outputs a genotype-phenotype mapping highlighting perturbation subspaces.

2. The computer-implemented method of claim 1, wherein the perturbation subspaces comprise viable genetic modifications that lead to useful phenotypes.

3. The computer-implemented method of claim 1, wherein the perturbation subspaces exclude unviable genetic modifications that lead to non-useful phenotypes.

4. The computer-implemented method of claim 1, wherein the perturbation subspaces comprise useful phenotypes for a synthetic biology application and/or product.

5. The computer-implemented method of claim 1, wherein the genotype-phenotype mapping shows genetic distance between the single genetic modifications and the multiple genetic modifications.

6. The computer-implemented method of claim 5, wherein the genetic distance and the perturbations subspaces are used to select one or more genetic insults as a starting point to engineer cells in synthetic biology applications.

7. The computer-implemented method of claim 1, wherein the multiple gene modifications comprise a combination of the single gene modifications.

8. A computer program product for genotype-phenotype mapping for single and multiple genetic insults comprising:
one or more computer readable storage media, and program instructions collectively stored on one or more computer readable storage media, the program instructions comprising:
program instructions for training a deep learning neural network with cellular morphology features from single genetic modifications;
program instructions for testing the deep learning neural network with cellular morphology features from multiple genetic modifications; and
program instructions for the trained and tested deep learning neural network to input a link between cellular morphology features caused by the single gene modifications and cellular morphology features caused by the multiple gene modifications and output a genotype-phenotype mapping highlighting perturbation subspaces.

9. The computer program product of claim 8, wherein the perturbation subspaces comprise viable genetic modifications that lead to useful phenotypes.

10. The computer program product of claim 8, wherein the perturbation subspaces exclude unviable genetic modifications that lead to non-useful phenotypes.

11. The computer program product of claim 8, wherein the perturbation subspaces comprise useful phenotypes for a synthetic biology application and/or product.

12. The computer program product of claim 8, further comprising measuring genetic distance of the single and multiple genetic insults.

13. The computer program product of claim 8, wherein the genotype-phenotype mapping shows genetic distance between the single genetic modifications and the multiple genetic modifications.

14. The computer program product of claim 13, wherein the genetic distance and the perturbations subspaces are used to select one or more genetic insults as a starting point to engineer cells in synthetic biology applications.

15. The computer program product of claim 4, wherein the multiple gene modifications comprise a combination of the single gene modifications.

16. A method comprising:
inducing single gene modifications in a first portion of a cell sample and obtaining a first set of cell images;
inducing multiple gene modifications in a second portion of the cell sample and obtaining a second set of cell images;
extracting cellular morphology features from the first and second set of cell images;
training a deep learning neural network with the cellular morphology features from the first set of cell images;
testing the deep learning neural network with the cellular morphology features from the second set of cell images,
wherein the trained and tested neural network applies the cellular morphology features from the first and second set of images as input and provides genotype-phenotype mapping highlighting perturbation subspaces.

17. The method of claim 16, wherein the perturbation subspaces comprise viable genetic modifications that lead to useful phenotypes.

18. The method of claim 16, wherein the perturbation subspaces exclude unviable genetic modifications that lead to non-useful phenotypes.

19. The method of claim 16, wherein the perturbation subspaces comprise useful phenotypes for a synthetic biology application and/or product.

20. The method of claim 16, further comprising measuring genetic distance of the single and multiple genetic insults.

21. The method of claim 16, wherein the genotype-phenotype mapping shows genetic distance between the single genetic modifications and the multiple genetic modifications.

22. The method of claim 21, wherein the genetic distance and the perturbations subspaces are used to select one or more genetic insults as a starting point to engineer cells in synthetic biology applications.

23. The method of claim 16, wherein the trained and tested neural network identifies links between cellular morphology features caused by the single gene modifications and cellular morphology features caused by the multiple gene modifications.

24. The method of claim 16, wherein the multiple gene modifications comprise a combination of the single gene modifications.
